(19) United States
(12) Patent Application Publication
(10) Pub. No.: US 2022/0401429 A1
(43) Pub. Date: Dec. 22, 2022

(54) USE OF ANAMORELIN TO TREAT CANCER-RELATED FATIGUE

(71) Applicant: Helsinn Healthcare SA, Lugano/Pazzallo (CH)

(72) Inventors: Sriram Yennu, Houston, TX (US); Eduardo Bruera, Houston, TX (US); Elena Palmesino, Lugano (CH); Ruben Giorgino, Allschwil (CH); Elizabeth Duus, Cape Coral, FL (US)

(21) Appl. No.: 17/838,308

(22) Filed: Jun. 13, 2022

Related U.S. Application Data
(60) Provisional application No. 63/212,148, filed on Jun. 18, 2021.

(51) Int. Cl. A61K 31/454

(52) U.S. Cl. CPC A61K 31/454

(57) ABSTRACT

Methods of using anamorelin to treat fatigue and increasing physical activity in defined cancerous patient populations.

### FIELD OF THE INVENTION

The present invention relates to anamorelin for the treatment of cancer-related fatigue and for improving subjective perception of physical activity in cancer patients.

### BACKGROUND OF THE INVENTION

Cancer related fatigue (“CRF”) is a frequent and serious consequence of cancer and cancer related treatment (Yennurajalingam, Frisbee-Hume et al. 2012, Yennurajalingam, Willey et al. 2012). As treatments for cancer improve and patients with cancer live longer, CRF is being recognized increasingly as an important factor affecting their quality of life (“QOL”).

Several strategies have been proposed for the management of CRF including physical activity, erythropoietin stimulating agents and psychostimulants, but with limited effect (Berger, Abernethy et al. 2010). Dexamethasone has been shown to produce clinically significant improvements in fatigue, and its efficacy has been attributed partly to its effect on inflammation due to its ability to reduce an inflammatory biomarker (C-reactive protein or “CRP”) in this population. However, due to dexamethasone's side-effect profile it can only be given for a short duration and is of limited utility in the treatment of this chronic condition (Yennurajalingam, Frisbee-Hume et al. 2013, Yennurajalingam and Bruera 2014).

Anamorelin, which is under development by Helsinn Healthcare (Lugano/Pazzallo Switzerland) for the treatment of cancer cachexia, has shown equivocal effects on fatigue (Currow and Abernethy 2014, Takayama, Katakami et al. 2016, Temel, Abernethy et al. 2016). Two phase III studies have been conducted with anamorelin (ROMANA 1 and ROMANA 2), and in both studies the investigators were unable to detect a significant effect on fatigue using the FACIT-F or strength measured by handgrip strength (Temel, Abernethy et al. 2016).

A pooled post-hoc analysis of the ROMANA 1 and ROMANA 2 studies showed that anamorelin was effective to treat fatigue and improve handgrip strength in patients with a body mass index (“BMI”) less than 20 kg/m² but ineffective at higher BMIs (Currow, Temel et al. 2016 (a)). Very little else has been done to identify patients whose fatigue would benefit from anamorelin intervention. While inflammation is commonly correlated with fatigue, phase III studies with anamorelin have shown that patients with CRP >10 mg/L respond as well as patients with CRP <10 mg/L in terms of lean body mass, FAACT Anorexia/Cachexia Scale [A/CS], and other clinical outcomes (Currow, Temel et al. 2016(b)).

What is needed are studies to better characterize patients who might benefit from anamorelin, particularly in terms of fatigue and ability and/or willingness to expend energy.

### SUMMARY OF INVENTION

Even though Temel and Currow 2016(a) reported in their analysis of ROMANA 1 and 2 that anamorelin was not effective against fatigue and handgrip strength in patients having a body mass index greater than 20 kg/m², the inventors have unexpectedly discovered that anamorelin is effective against fatigue and self-reported measures of strength in many patients that have a body mass index greater than 20 kg/m², particularly in cancer patients characterized by systemic inflammation (e.g. CRP ≥3 mg/L) and fatigue (e.g. a fatigue score on the FACIT-F subscale ≤34). This result is even more surprising considering that Temel and Currow 2016(b) reported that anamorelin's efficacy was unaffected by systemic inflammation.

Thus, in a first principal embodiment the invention provides a method of treating cancer-related fatigue in a patient in need thereof comprising orally administering to the patient a therapeutically effective amount of anamorelin, wherein the patient has: (a) a fatigue score on the FACIT-F subscale of ≤34; (b) a C-reactive protein concentration of ≥3 mg/L; and (c) cancer. The results with anamorelin are especially pronounced in non-depressed patients and in patients not suffering from anemia, and in preferred embodiments the patient has a hemoglobin concentration ≥9 g/dL).

The inventors have further discovered that anamorelin can improve subjective perception of physical activity in cancer patients, in spite of previous teachings that anamorelin was not effective to increase handgrip strength. Thus, in a second principal embodiment the invention provides a method of increasing subjective perception of physical activity in a cancer patient in need thereof comprising orally administering to the patient a therapeutically effective amount of anamorelin. The method preferably comprises an increase of physical activity on the Godin-Shephard Leisure-Time Physical Activity Questionnaire.

The methods also apply generally to any cancer patient suffering from fatigue. Thus, in a third principal embodiment the invention provides a method of treating cancer-related fatigue in a patient in need thereof comprising orally administering to the patient a therapeutically effective amount of anamorelin. The method preferably comprises treatment of cancer-related fatigue on the FACIT-F subscale.

Additional advantages of the invention are set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying drawings, which are incorporated in and constitute a part of this specification, illustrate several embodiments of the invention and together with the description serve to explain the principles of the invention.

FIG. 1 plots a linear mixed model adjusted prediction of FACIT-F scores (95% CI) over time from the study reported in Example 1.

### DETAILED DESCRIPTION

### Definitions and Use of Terms

As used in the specification and claims, the singular forms a, an, and the include plural references unless the context clearly dictates otherwise. For example, the term “a specification” refers to one or more specifications for use in the presently disclosed methods and systems. “An ingredient” includes mixtures of two or more such ingredients, and the like. The word “or” or like terms as used herein means any one member of a particular list and also includes any combination of members of that list.

As used in this specification and in the claims which follow, the word “comprise” and variations of the word, such as “comprising” and “comprises,” means “including but not limited to,” and is not intended to exclude, for example, other additives, components, integers or steps. When an element is described as comprising one or a plurality of components, steps or conditions, it will be understood that the element can also be described as “consisting of” or “consisting essentially of” the component, step or condition, or the plurality of components, steps or conditions.

“Therapeutically effective amount” means that amount which, when administered to a human for supporting or affecting a metabolic process, or for treating or preventing a disease, is sufficient to cause such treatment or prevention of the disease or supporting or affecting the metabolic process. In any of the embodiments or subembodiments of this invention, a therapeutically effective amount preferably comprises 100 mg of anamorelin when administered orally as the free base once daily.

When “drug therapy,” “drug administration,” and like terms are used herein, it will be understood that the therapy can be accomplished through any suitable route of administration using any acceptable dosage form, and that the drug can be administered as the free base, a salt, or an ester or other prodrug moiety.

When used herein the term “about” will compensate for variability allowed for in the pharmaceutical industry and inherent in products in this industry, such as differences in product strength due to manufacturing variation and time-induced product degradation, as well as differences due to waters of hydration and different salts. The term allows for any variation, which in the practice of good manufacturing practices, would allow the product being evaluated to be considered therapeutically equivalent or bioequivalent in humans to the recited strength of a claimed product. In one embodiment the term allows for any variation within 5% of the recited specification or standard. In one embodiment the term allows for any variation within 10% of the recited specification or standard.

When published test methodologies and diagnostic instruments are referred to herein, it will be understood that the test methodology or diagnostic instrument is performed based on the version in effect on Jan. 1, 2021, unless otherwise stated to the contrary herein.

When ranges are expressed herein by specifying alternative upper and lower limits of the range, it will be understood that the endpoints can be combined in any manner that is mathematically feasible. Thus, for example, a range of from 50 or 80 to 100 or 70 can alternatively be expressed as a series of ranges of from 50 to 100, from 50 to 70, and from 80 to 100. When a series of upper bounds and lower bounds are related using the phase “and” or “or”, it will be understood that the upper bounds can be unlimited by the lower bounds or combined with the lower bounds, and vice versa. Thus, for example, a range of greater than 40% and/or less than 80% includes ranges of greater than 40%, less than 80%, and greater than 40% but less than 80%. Unless otherwise specified by the term “between,” the boundaries of the range (lower and upper ends of the range) are included in the claimed range.

When an element of a process or thing is defined by reference to one or more examples, components, properties or characteristics, it will be understood that any one or any combination of those components, properties or characteristics can also be used to define the matter at issue. This might occur, for example, when specific examples of an element are recited in a claim (as in a Markush grouping), or an element is defined by a plurality of characteristics. Thus, for example, if a claimed system comprises element A defined by elements A1, A2 and A3, in combination with element B defined by elements B1, B2 and B3, the invention will also be understood to cover a system defined by element A without element B, a system in which element A is defined by elements A1 and A2 in combination with element B defined by elements B2 and B3, and all other possible permutations.

In the context of the present invention insofar as it relates to any of the disease conditions recited herein, the term “treatment” means to reduce the occurrence of a symptom or condition, or to relieve or alleviate at least one symptom associated with such condition, or to slow or reverse the progression of such condition, or to manage or affect the metabolic processes underlying such condition. Within the meaning of the present invention, the term also denotes to arrest, or to “prevent,” i.e. to delay the onset (i.e., the period prior to clinical manifestation of a disease) and/or reduce the risk of developing or worsening a disease. When a person with the condition is affirmatively recited, the term will be understood to require relief or alleviation of at least one symptom associated with the condition.

Biomarker test assays —unless otherwise indicated herein, all biomarker test assays referred to herein are performed in accordance with standard procedures employed during the 2001-2002 cycle of the National Health and Nutrition Examination Survey.

### Discussion of Principal Embodiments

In a first principal embodiment the invention provides a method of treating cancer-related fatigue in a patient in need thereof comprising orally administering to the patient a therapeutically effective amount of anamorelin, wherein the patient has: (a) a fatigue score on the FACIT-F subscale of ≤34; (b) a C-reactive protein concentration of ≥3 mg/L; (c) cancer; (d) no evidence of depression; and (e) a hemoglobin level ≥9 g/dL.

In a second principal embodiment the invention provides a method of increasing subjective perception of physical activity in a cancer patient in need thereof comprising orally administering to the patient a therapeutically effective amount of anamorelin. The method preferably comprises an increase of energy expenditure on the Godin-Shephard Leisure-Time Physical Activity Questionnaire.

In a third principal embodiment the invention provides a method of treating cancer-related fatigue in a patient in need thereof comprising orally administering to the patient a therapeutically effective amount of anamorelin. The method preferably comprises treatment of cancer-related fatigue on the FACIT-F subscale.

### Discussion of Subembodiments

The invention can further be understood with reference to various subembodiments which can modify any of the principal embodiments. It will be understood that these subembodiments can be combined in any manner that is both mathematically and physically possible to create additional subembodiments, which in turn can modify any of the principal embodiments.

In any of the embodiments of the present invention, the patient preferably suffers from the systemic inflammation originating from cancerous processes. I.e., the patient preferably does not have an infection or autoimmune disorder or other non-cancerous cause of systemic inflammation. The patient also preferably suffers from fatigue, preferably as measured on the FACIT-F subscale.

Thus, in various subembodiments the patient has (a) a fatigue score on the FACIT-F subscale of ≤34; and/or (b) a C-reactive protein concentration of ≥3 mg/L. In other embodiments that patient has: (a) a fatigue score on the FACIT-F subscale of ≤34; (b) a C-reactive protein concentration of ≥3 mg/L; (c) cancer; (d) no evidence of depression; and/or (e) a hemoglobin level ≥9 g/dL.

Various cut-points can be used to measure the patient's fatigue. Thus, in various embodiments the patient has a fatigue score on the FACIT-F subscale of ≤30, ≤26, ≤22, or ≤18. In like manner, various cut-points can be used to measure the patient's systemic inflammation. Thus, in various alternative or additional embodiments, the patient has a C-reactive protein concentration of ≥5 mg/L; ≥10 mg/L, ≥15 mg/L, or ≥20 mg/L.

The patient also can be defined based on various clinical parameters. Thus, in one subembodiment the patient has advanced cancer. In another subembodiment the patient has advanced cancer other than non-small cell lung cancer. In still another subembodiment the patient has metastatic or recurrent incurable solid tumors. In yet another subembodiment, the patient is on chemotherapy, immunotherapy, or radiation therapy for cancer.

The patient also can be characterized based on various measures of weight and body mass. Thus, in another subembodiment the patient has an unintentional weight loss of from 2% to 15% within the previous 12 months. In one subembodiment the patient has an unintentional body weight loss of less than 5%, 4%, or 3% in the previous 12 months. In another subembodiment the patient has a BMI greater than 20 kg/m², greater than 22.5 kg/m², or greater than 25 kg/m².

Improvements from the methods of the present invention can be measured using various clinical instruments. Thus, in one subembodiment the method results in a clinically meaningful improvement on the FACIT-F subscale. In another subembodiment the method results in a clinically meaningful improvement on the Godin-Shephard Leisure-Time Physical Activity Questionnaire. In other subembodiments the method results in a clinically meaningful improvement on a scale selected from PROMIS-fatigue, MFSI-SF-general, and FAACT-anorexia.

The patients of the current invention can further be characterized using various biochemical measurements. Thus, in one subembodiment the patient has a hemoglobin level of greater than 9, 10, 11, or 12 g/dL. In yet another subembodiment the patient has: (a) an albumin concentration in the normal range of from 3.4 to 5.4 g/dL, (b) a fasting plasma glucose concentration of from 70 to 140 mg/dL; and/or (c) an IGF-1 concentration of from 75 to 150 ng/mL.

The methods of the present invention are preferably practiced in combination with physical exercise and nutrition management.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention.

### Example 1: Controlled Study of Anamorelin for Cancer Related Fatigue

This study was undertaken to isolate and evaluate anamorelin's effect on fatigue, energy expenditure, and other side effects of cancer in a cancer-stricken population, by including in the study population only those patients suffering significant levels of fatigue and inflammatory activity, excluding from the study population confounding factors such as depression and anemia, and controlling for a consistent level of nutrition and physical exercise.

In this single arm study, 38 patients were intended for treatment with anamorelin for 6 weeks with an optional extension phase of 6 more weeks based on tolerability and safety and patient willingness. Patients received anamorelin 100 mg tablets and physical activity for 6 weeks for the intervention phase. Patients took 1 tablet orally while fasting (at least 1 hour before a meal).

### Major Inclusion Criteria

diagnosis of advanced cancer (metastatic or recurrent incurable solid tumors excluding prostate cancer)
fatigue on FACIT-F subscale of ≤34 on a 0 to 52 scale [in which 52=no fatigue and 0=worst possible fatigue]
CRP ≥3 mg/L* in the absence of any other more likely cause of increased CRP like an infection or an autoimmune disorder
unintentional weight loss of from 2% to 15% at any time within the previous 12 months
No evidence of moderate to severe depression as determined by a HADS depression score of ≤13
Hemoglobin level ≥9 g/dL

### Major Exclusion Criteria

Regularly engaged in moderate or vigorous-intensity exercise for at least 5 times a week
Inability to complete the baseline assessment forms or to understand the recommendations for participation in the study.
Pregnant or lactating women, childbearing age women who are not on birth control.
Uncontrolled diabetes mellitus (Fasting Blood Sugar >200 mg/dL) at screening
Patients with prostate cancer
   *CRP is a biomarker for fatigue in palliative care patients hence was used as eligibility criteria. Recent studies suggest 3 mg/dL as an ideal cut off for significant inflammation (Amano, Maeda et al. 2017, Paulsen, Laird et al. 2017).

### Primary Endpoint

A primary objective of the study was to examine the effects of anamorelin and standardized physical activity and nutritional counseling on changes in the Functional Assessment of Cancer Illness Therapy-Fatigue (FACIT-F) subscale score at Day 43±3 days compared to baseline in patients with advanced cancer receiving oral anamorelin 100 mg daily and standardized physical activity and nutritional counseling.

FACIT-F is a well-validated QOL instrument in cancer patients (Cella, Tulsky et al. 1993). The 13-item fatigue subscale is a patient-rated assessment of intensity of fatigue and its related symptoms on a scale of 0 to 4. This scale has been shown to have strong internal consistency (α=0.93−0.95), sensitivity of 0.92, and specificity of 0.6923 (Cella, Tulsky et al. 1993).

### Secondary Endpoints

Secondary objectives of the study were to examine the effects of anamorelin and standardized physical activity and nutritional counseling on Health-related quality of life and patient reported outcomes as measured by The Multidimensional Fatigue Symptom Inventory-Short Form[MFSI-SF], PROMIS-Fatigue, Hospital Anxiety Depression Scale (HADS), Edmonton Symptom Assessment Scale (ESAS), Functional Assessment of Cancer Therapy (FACT-G), and its Functional Assessment of Anorexia/Cachexia Treatment (FAACT) subscale in these patients.

MFSI-SF consists of 30 items designed to assess the multidimensional nature of fatigue(Stein, Jacobsen et al. 2004). Ratings are summed to obtain scores for 5 subscales (general fatigue, physical fatigue, emotional fatigue, mental fatigue, and vigor).

PROMIS measures key symptoms and health concepts applicable to advanced cancer, enabling efficient and interpretable clinical trial research and clinical practice application of patient-reported outcomes (PROs) (Rothrock, Hays et al. 2010, Yost, Eton et al. 2011). The PROMIS fatigue measure used in the study was found to be highly correlated with the legacy measures (Barsevick, Irwin et al. 2013).

HADS: Depression symptoms were assessed at the time of screening for the patient to be enrolled in the protocol using the 14-item HADS questionnaire. This questionnaire asks patients to underline the statement that most closely matches how they have been feeling in the previous week. This questionnaire has been found to be valid and reliable in a number of clinical situations and has been widely used in medically ill patients (Johnston, Pollard et al. 2000).

The FACT-G is a 27-item compilation of general questions divided into four primary domains: Physical Well-Being, Social/Family Well-Being, Emotional Well-Being, and Functional Well-Being. It is appropriate for use in patients with any form of cancer, and extensions of it have been used and validated in other chronic illness conditions as well (Cella, Tulsky et al. 1993, Webster, Cella et al. 2003). The FACT-G is the first 2 pages of FACIT-F.

The FAACT-Anorexia/Cachexia subscale is a 12-item symptom-specific subscale designed to measure patients' additional concerns about their anorexia/cachexia during the previous 7 days. The FAACT has internal consistency and a reliability coefficient (Cronbach's alpha) of 0.88 for the 12 components (Ribaudo, Cella et al. 2000). Patients rate the intensity of anorexia/cachexia and its related symptoms on a 0-4 scale like that used in the FACIT-F.

The ESAS measures 10 common symptoms in the past 24 hours (pain, fatigue, nausea, depression, anxiety, drowsiness, shortness of breath, appetite, sleep, and feeling of well-being); This questionnaire has been found valid and reliable in cancer populations (Bruera, Kuehn et al. 1991).

### Exploratory Endpoints

Exploratory objectives of the study were to explore the effects of anamorelin and standardized physical activity and nutritional counseling on muscle function (as measured by the 30 second chair stand test, 6 minute walk test, day time activity (accelerometer), body composition (as measured by INBODY), and Resting Energy Expenditure (measured by indirect calorimetry)

Measures: We used the 30 second chair stand test, six-minute walk test, and an accelerometer. A self-report exercise questionnaire, the Godin Leisure-Time physical activity questionnaire, was used to complement the objective measures. IM Systems-three dimensional accelerometers (“Biotrainer-Pro”) was used to objectively measure physical activity.

Physical Performance Tests: The 30 second chair stand test, 6-minute walk test, and Godin Leisure-Time physical activity questionnaire were administered at baseline and on Day 43. The 30 second sit-to-stand task assessed lower body strength (Rikli and Jones 1999).

In the six-minute walk test, participants were asked to walk as fast and as far as they can for six minutes, and the distance walked was measured. The six-minute walk test has high test-retest and inter-tester reliability, and its validity is supported by correlations with self-report measures of fatigue and functional status (Simmonds 2002). The six minute walk test was performed as per the ATS guidelines:

(https://www.thoracic. org/statements/resources/pfet/sixminute.pdf.)

The Godin Leisure-Time physical activity questionnaire asks participants how many times per week on average they participate in strenuous, moderate and mild exercises for more than 15 minutes during their free time. It also asks how often the participant engages in regular activity long enough to work up a sweat during their leisure time in a typical week (often, sometimes, never/rarely) (Gionet and Godin 1989; Godin and Shephard 1985).

Body Composition: All patients were assessed for lean body mass, skeletal muscle mass, extracellular water/total body water body, fat mass, body fat mass, and whole body phase angle evaluation (using the InBody body composition scale) (Dalal, Hui et al. 2012, Del Fabbro, Parsons et al. 2012, Yennurajalingam, Willey et al. 2012, Hui, Bansal et al. 2014). Resting energy expenditure was assessed at baseline and Day 43±3 days, and at the end of 6 weeks of the extension phase.

### Exercise Prescription:

Exercise was prescribed based on the American College of Sports Medicine (ACSM) exercise recommendations for cancer survivors to ensure safety and maximal benefit. (Schmitz, Courneya et al. 2010). The exercise intervention includes (a) resistance training 3 days/week and (b) moderate intensity walking for up to 150 minutes per week. The resistance exercise program was designed to strengthen the major muscles of the lower body, including the quadriceps, hamstrings, gluteus maximus, and hip flexor group. Exercises included (without limitation) squats, lunges, leg extensions, leg curls, and hip extensions. Resistance tubes were used as the mode of resistance. Resistance exercise sessions were completed 3 days a week, allowing at least 48 hours between each session. The participant began with 1 set of 10 to 12 repetitions at the lightest resistance progressing to 2 sets of 12 repetitions as exercise tolerance increases. Resistance was increased as the participant's endurance and strength progresses. For the graded resistance program the individual began with a lighter resistance and progressed to heavier resistance once a level was mastered. The participant began with 2 sets of 12 repetitions at the next established intensity level.

Because the level of aerobic fitness varied among participants, the frequency and duration of the walking program was established based on the exercise physiologist's assessment of the participant's baseline aerobic fitness level (six-minute walk test). For example, the recommendation may be to walk 10 minutes 1-3 times a day or up to a 30-minute walk once a day at a moderate intensity level. Participants worked toward the goal of 150 minutes of moderate intensity walking per week.

### Nutrition Counseling

All patients enrolled in this study received at baseline and day 21(±3) nutritional support by a dietitian, with a goal of achieving 1.5× Resting Energy Expenditure (REE) estimated by the Mifflin St. Jeor method (Mifflin, St Jeor et al. 1990). Frequent small meals that are calorie dense were recommended. Commercially available amino acids preparations rich in arginine, glutamine, and leucine-related products such as Beta hydroxyl Beta methylbutyrate, were prescribed when advised by the dietitian to assist patients achieve calorie goals and maintain lean body mass (Molfino, Gioia et al. 2013).

### Results

Of the 45 patients enrolled, 28 were evaluable at Day 43. The median age was 64 years; 64% were males, and 82.1% white. The mean (SD) for FACIT-F subscale improvement from baseline was 4.89 (±13.07), P=0.058, MFSI-SF (G) −3.46 (±6.86), P=0.013, PROMIS-fatigue-4.14 (±7.88), P=0.010, FAACT ACS 3.48 (±8.13), P=0.035. Godin Leisure-Time physical activity questionnaire 7.41 (±16.50), P=0.038. Weight (kg) 1.81 (±2.63), P=0.005, and Lean Body Mass 1.54 (±1.85), P=0.001, IGF-1 36.50 (±48.76), P=0.075. There was no significant improvement in the physical performance outcomes and mean daytime activity (actigraphy activity measure). No severe (≥grade 3) adverse events related to the study drug were reported. Detailed results are reported in Tables 1 and 2.

TABLE 1
Changes in Cancer Related Fatigue Scores

| | Day 15 (N = 28) | | Day 29 (N = 28) | | Day 43 (N = 28) | | Day 86 (N = 8) | |
|---|---|---|---|---|---|---|---|---|
| | Mean | P-value* | Mean | P-value* | Mean | P-value* | Mean | P-value* |
| | (±SD), | (Cohen's | (±SD), | (Cohen's | (±SD), | (Cohen's | (±SD), | (Cohen's |
| | CI | D) | CI | D) | CI | D) | CI | D) |
| FACIT-F | 3.89 (±8.71), | 0.025 | 6.32 (±9.53), | 0.002 | 4.89 (±13.07), | 0.058 | 8.13 (±12.62), | 0.12 |
| Fatigue | (0.52, 7.27) | (0.45) | (2.63, 10.02) | (0.66) | (−0.18, 9.96) | (0.37) | (−2.43, 18.68) | (0.64) |
| Subscale | | | | | | | | |
| ESAS- | −0.68 (±2.87), | 0.22 | −0.75 (±2.70), | 0.15 | −0.81 (±2.87), | 0.15 | −1.25 (±2.43), | 0.16 |
| Fatigue | (−1.79, 0.43) | (−0.24) | (−1.80, 0.30) | (−0.28) | (−1.95, 0.32) | (−0.28) | (−3.29, 0.79) | (−0.51) |

MFSI-SF

| | | | | | | | | |
|---|---|---|---|---|---|---|---|---|
| General | −2.25 (±4.53), | 0.014 | −4.18 (±5.74), | 0.001 | −3.46 (±6.86), | 0.013 | −4.00 (±6.28), | 0.09 |
| fatigue | (−4.01, −0.49) | (−0.50) | (−6.40, −1.95) | (−0.73) | (−6.21, −0.81) | (−0.51) | (−9.25, 1.25) | (−0.64) |
| Physical | −0.71 (±3.69), | 0.31 | −0.50 (±4.43), | 0.56 | −0.39 (±5.03), | 0.68 | −1.88 (±3.98), | 0.20 |
| fatigue | (−2.15, 0.72) | (−0.19) | (−2.22, 1.22) | (−0.11) | (−2.34, 1.56) | (−0.08) | (−5.20, 1.45) | (−0.47) |
| Emotional | −0.43 (±2.20), | 0.31 | 0.14 (±3.15), | 0.81 | 1.04 (±4.36), | 0.22 | −0.88 (±2.42), | 0.29 |
| fatigue | (−1.28, 0.43) | (−0.19) | (−1.08, 1.36) | (0.05) | (−0.65, 2.73) | (0.24) | (−2.90, 1.15) | (−0.36) |
| Mental | −0.93 (±2.57), | 0.07 | −1.39 (±2.90), | 0.017 | −0.36 (±4.12), | 0.65 | −2.25 (±3.62), | 0.11 |
| fatigue | (−1.92, 0.07) | (−0.36) | (−2.52, −0.27) | (−0.48) | (−1.95, 1.24) | (−0.09) | (−5.27, 0.77) | (−0.62) |
| Vigor | 0.46 (±2.89), | 0.40 | 0.39 (±3.46), | 0.55 | −1.11 (±5.28), | 0.28 | 2.75 (±3.01), | 0.08 |
| | (−0.65, 1.58) | (0.16) | (−0.95, 1.73) | (0.11) | (−3.15, 0.94) | (−0.21) | (0.23, 5.27) | (0.91) |
| MFSI-SF | −4.79 (±9.59), | 0.017 ^{a} | −6.32 (±12.41), | 0.012 | −2.07 (±17.59), | 0.54 | −11.75 (±14.22), | 0.050 |
| Total | (−8.50, −1.07) | (−0.50) | (−11.53, −1.51) | (−0.51) | (−8.89, 4.75) | (−0.12) | (−23.64, 0.14) | (−0.83) |

PROMIS

| | | | | | | | | |
|---|---|---|---|---|---|---|---|---|
| PROMIS- | −2.74 (±6.12), | 0.025 | −3.64 (±8.22), | 0.027 | −4.14 (±7.88), | 0.010 | −6.36 (±6.32), | 0.025 |
| Fatigue | (−5.11, −0.37) | (−0.45) | (−6.82, −0.45) | (−0.44) | (−7.19, −1.08) | (−0.53) | (−11.65, −1.08) | (−1.01) |

Abbreviations: FACIT-F, Functional Assessment of Chronic Illness Therapy-Fatigue subscale; ESAS, Edmonton Symptom Assessment Scale; MFSI-SF, Multidimensional Fatigue Symptom Inventory-Short Form; PROMIS, Patient-Reported Outcomes Measurement Information System -Fatigue. SD, Standard Deviation; CI, Confidence Interval, Lower and Upper Confidence Interval;
*P-values are derived using Paired sample t-test; all values were derived using Wilcoxon Signed Rank Test.

TABLE 2
Changes in Other Measures

| | Day 15 (N = 28) | | Day 29 (N = 28) | | Day 43 (N = 28) | | Day 86 (N = 8) | |
|---|---|---|---|---|---|---|---|---|
| | Mean | P-value* | Mean | P-value* | Mean | P-value* | Mean | P-value* |
| | (±SD), | (Cohen's | (±SD), | (Cohen's | (±SD), | (Cohen's | (±SD), | (Cohen's |
| | CI | D) | CI | D) | CI | D) | CI | D) |
| FACT-G | 0.59 (±9.17), | 0.74 | 1.31 (±8.97), | 0.45 | −1.10 (±10.71), | 0.59 | 4.54 (±7.65), | 0.18 |
| Total | (−2.96, 4.15) | (0.06) | (−2.17, 4.79) | (0.15) | (−5.26, 3.05) | (−0.10) | (−1.85, 10.94) | (0.59) |
| FAACT- | 1.70 (±4.89), | 0.082 | 4.53 (±5.54), | <0.001 | 3.48 (±8.13), | 0.035^{a} | −5.86 (±3.72), | 0.018 |
| A/CS Total | (−0.23, 3.64) | (0.35) | (2.34, 6.72) | (0.82) | (0.27, 6.70) | (0.43) | (2.42, 9.29) | (1.58) |
| ESAS | −0.43 (±3.06), | 0.47 | −1.04 (±3.19), | 0.097 | −0.56 (±3.07), | 0.35 | −0.13 (±3.09), | 0.57 |
| Appetite | (−1.62, 0.76) | (−0.14) | (−2.27, 0.20) | (−0.32) | (−1.77, 0.66) | (−0.18) | (−2.46, 2.71) | (0.04) |
| PSQI | | | −0.09 (±4.18), | 0.94 | −0.09 (±2.12), | 0.89 | −0.50 (±2.95), | 1.00 |
| (N = 15) | | | (−2.90, 2.72) | (−0.02) | (−1.51, 1.33) | (−0.04) | (−3.60, 2.60) | (−0.17) |
| Godin | | | | | 7.41 (±16.50), | 0.038 ^{a} | −0.67 (±6.35), | 1.00 |
| Leisure | | | | | (0.09, 14.72) | (0.45) | (−16.44, 15.11) | (−0.10) |
| Test Total | | | | | | | | |

Global Symptom Evaluation

| | |
|---|---|
| Better N (%) | 13 (52%) |
| About the same N (%) | 9 (36%) |
| Worse N (%) | 3 (12%) |

Abbreviations: FACT-G, Functional Assessment of Cancer Therapy-General; FAACT-A/CS: Functional Assessment of Anorexia Cachexia Therapy-Anorexia/Cachexia Scale; ESAS, Edmonton Symptom Assessment System; PSQI, Pittsburgh Sleep Quality Index; SD, Standard Deviation; CI, Confidence Interval, Lower and Upper Confidence Interval;
*P values are derived using Paired sample t-test;
^{a} P values were derived using Wilcoxon Signed Rank Test.

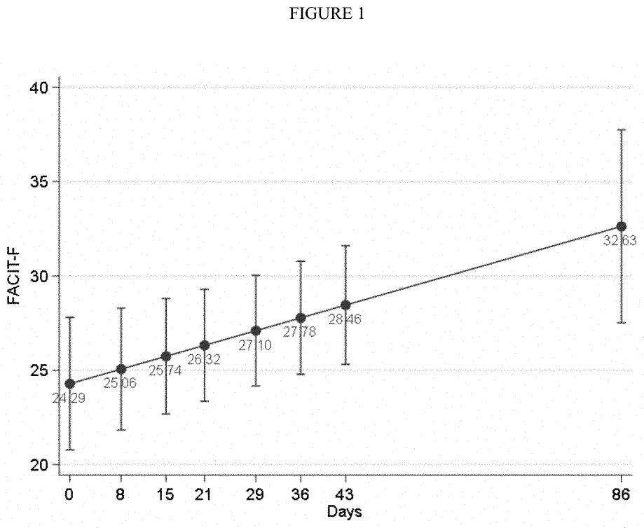

FIG. 1 shows FACIT-F scores (95% CI) at various time points. As seen in FIG. 1, using linear mixed models, a significant time effect was found with FACIT-F increasing on average of 0.11 (95% CI: 0.03-0.19; p=0.007) units per day. Similarly, if time was included as a categorical variable in the model, then the 43-day measure had a significant increase from baseline of 4.89 (95% CI: 0.67-9.11; p=0.023).

Several additional results should be highlighted from this study. For example, the effect size (0.37) for improvement in CRF was significantly larger in this study compared to previous studies where effect sizes were 0.16 in ROMANA 1, 0.008 in ROMANA 2 study, and 0.057 in a study by Katakami and Uchino et al. 2018. The positive outcomes in the Godin Leisure score were also notable given that this score assesses patient reported physical activity, and until this study very little effect had been shown in physical strength testing.

### CONCLUSION

The use of the combination of Anamorelin with physical activity and nutritional counseling was associated with improvement of subjective patient reported outcomes for CRF (FACIT-F fatigue, PROMIS-fatigue, MFSI-SF—general), activity (Godin Leisure-Time), anorexia (FAACT), body composition, and IGF-1 levels.

### REFERENCES

Amano, K., I. Maeda, T. Morita, M. Baba, T. Miura, T. Hama, I. Mori, N. Nakajima, T. Nishi, H. Sakurai, S. Shimoyama, T. Shinjo, H. Shirayama, T. Yamada, S. Ono, T. Ozawa, R. Yamamoto, N. Yamamoto, H. Shishido and H. Kinoshita (2017). “C-reactive protein, symptoms and activity of daily living in patients with advanced cancer receiving palliative care.” J Cachexia Sarcopenia Muscle 8(3): 457-465.
Barsevick, A. M., M. R. Irwin, P. Hinds, A. Miller, A. Berger, P. Jacobsen, S. Ancoli-Israel, B. B. Reeve, K. Mustian, A. O'Mara, J. S. Lai, M. Fisch, D. Cella and M. National Cancer Institute Clinical Trials Planning (2013). “Recommendations for high-priority research on cancer-related fatigue in children and adults.” J Natl Cancer Inst 105(19): 1432-1440.
Berger, A. M., A. P. Abernethy, A. Atkinson, A. M. Barsevick, W. S. Breitbart, D. Cella, B. Cimprich, C. Cleeland, M. A. Eisenberger, C. P. Escalante, P. B. Jacobsen, P. Kaldor, J. A. Ligibel, B. A. Murphy, T. O'Connor, W. F. Pirl, E. Rodler, H. S. Rugo, J. Thomas and L. I. Wagner (2010). “NCCN Clinical Practice Guidelines Cancer-related fatigue.” J Natl Compr Canc Netw 8(8): 904-931.
Bruera, E., N. Kuehn, M. J. Miller, P. Selmser and K. Macmillan (1991). “The Edmonton Symptom Assessment System (ESAS): a simple method for the assessment of palliative care patients.” J Palliat Care 7(2): 6-9.
Cella, D., D. T. Eton, J. S. Lai, A. H. Peterman and D. E. Merkel (2002). “Combining anchor and distribution-based methods to derive minimal clinically important differences on the Functional Assessment of Cancer Therapy (FACT) anemia and fatigue scales.” J Pain Symptom Manage 24(6): 547-561.
Cella, D. F., D. S. Tulsky, G. Gray, B. Sarafian, E. Linn, A. Bonomi, M. Silberman, S. B. Yellen, P. Winicour, J. Brannon and et al. (1993). “The Functional Assessment of Cancer Therapy scale: development and validation of the general measure.” J Clin Oncol 11(3): 570-579.
Currow, D. C., Temel, J. S., Giorgino, R., Abernethy A. P., Friend, J., Fearon, K. (2016). “Anamorelin in cachectic patients with non-small cell lung cancer (NSCLC) and low BMI (<20 kg/m²): A pooled efficacy data analysis of two phase 3 studies.” J Clin Oncol 34 (suppl; abstr 10119) (Currow, Temel et al. 2016(a)).
D. Currow, J. Temel, A. Abernethy, J. Friend, R. Giorgino, K. C. Fearon (2016). “Anamorelin in cachectic patients with non-small cell lung cancer (NSCLC) and acute phase protein reaction (APPR): Pooled analysis of two phase 3 trials (ROMANA 1 and ROMANA 2).” Annals of Oncology 27 (Supplement 6): vi497-vi521 (Currow, Temel et al. 2016(b))
Currow, D. C. and A. P. Abernethy (2014). “Anamorelin hydrochloride in the treatment of cancer anorexia-cachexia syndrome.” Future Oncol 10(5): 789-802.
Dalal, S., D. Hui, L. Bidaut, K. Lem, E. Del Fabbro, C. Crane, C. C. Reyes-Gibby, D. Bedi and E. Bruera (2012). “Relationships among body mass index, longitudinal body composition alterations, and survival in patients with locally advanced pancreatic cancer receiving chemoradiation: a pilot study.” J Pain Symptom Manage 44(2): 181-191.
Del Fabbro, E., H. Parsons, C. L. Warneke, K. Pulivarthi, J. K. Litton, R. Dev, S. L. Palla, A. Brewster and E. Bruera (2012). “The relationship between body composition and response to neoadjuvant chemotherapy in women with operable breast cancer.” Oncologist 17(10): 1240-1245.
Gionet N J, Godin G. Self-reported exercise behavior of employees: a validity study. J Occup Med. 1989; 31: 969-973.
Godin G, Shephard R J. A simple method to assess exercise behavior in the community. Can J Appl Sport Sci. 1985; 10: 141-146.
Hui, D., S. Bansal, M. Morgado, R. Dev, G. Chisholm and E. Bruera (2014). “Phase angle for prognostication of survival in patients with advanced cancer: preliminary findings.” Cancer 120(14): 2207-2214.
Johnston, M., B. Pollard and P. Hennessey (2000). “Construct validation of the hospital anxiety and depression scale with clinical populations.” J Psychosom Res 48(6): 579-584.
N. Katakami, J. Uchino, T. Yokoyama, T. Naito, M. Kondo, K. Yamada, H. Kitajima, K. Yoshimori, K. Sato, H. Saito, K. Aoe, T. Tsuji, Y. Takiguchi, K. Takayama, N. Komura, T. Takiguchi, and K. Eguchi (2018). “Anamorelin (ONO-7643) for the Treatment of Patients With Non—Small Cell Lung Cancer and Cachexia: Results From a Randomized, Double-Blind, Placebo-Controlled, Multicenter Study of Japanese Patients (ONO-7643-04).” Cancer Feb. 1, 2018; 606-616.
Mifflin, M. D., S. T. St Jeor, L. A. Hill, B. J. Scott, S. A. Daugherty and Y. O. Koh (1990). “A new predictive equation for resting energy expenditure in healthy individuals.” Am J Clin Nutr 51(2): 241-247.
Molfino, A., G. Gioia, F. Rossi Fanelli and M. Muscaritoli (2013). “Beta-hydroxy-beta-methylbutyrate supplementation in health and disease: a systematic review of randomized trials.” Amino Acids 45(6): 1273-1292.
Paulsen, O., B. Laird, N. Aass, T. Lea, P. Fayers, S. Kaasa and P. Klepstad (2017). “The relationship between pro-inflammatory cytokines and pain, appetite and fatigue in patients with advanced cancer.” PLoS One 12(5): e0177620.
Ribaudo, J. M., D. Cella, E. A. Hahn, S. R. Lloyd, N. S. Tchekmedyian, J. Von Roenn and W. T. Leslie (2000). “Re-validation and shortening of the Functional Assessment of Anorexia/Cachexia Therapy (FAACT) questionnaire.” Qual Life Res 9(10): 1137-1146.
Rikli, R. E. and C. J. Jones (1999). “Development and validation of a functional fitness test for community-residing older adults.” Journal of Aging and Physical Activity 7(2): 129-161.
Rothrock, N. E., R. D. Hays, K. Spritzer, S. E. Yount, W. Riley and D. Cella (2010). “Relative to the general US population, chronic diseases are associated with poorer health-related quality of life as measured by the Patient-Reported Outcomes Measurement Information System (PROMIS).” J Clin Epidemiol 63(11): 1195-1204.
Simmonds, M. J. (2002). “Physical function in patients with cancer: psychometric characteristics and clinical usefulness of a physical performance test battery.” J Pain Symptom Manage 24(4): 404-414.
Takayama, K., N. Katakami, T. Yokoyama, S. Atagi, K. Yoshimori, H. Kagamu, H. Saito, Y. Takiguchi, K. Aoe, A. Koyama, N. Komura and K. Eguchi (2016). “Anamorelin (ONO-7643) in Japanese patients with non-small cell lung cancer and cachexia: results of a randomized phase 2 trial.” Support Care Cancer 24(8): 3495-3505.
Temel, J. S., A. P. Abernethy, D. C. Currow, J. Friend, E. M. Duus, Y. Yan and K. C. Fearon (2016). “Anamorelin in patients with non-small-cell lung cancer and cachexia (ROMANA 1 and ROMANA 2): results from two randomised, double-blind, phase 3 trials.” Lancet Oncol 17(4): 519-531.
Webster, K., D. Cella and K. Yost (2003). “The Functional Assessment of Chronic Illness Therapy (FACIT) Measurement System: properties, applications, and interpretation.” Health Qual Life Outcomes 1(1): 79.
Yennurajalingam, S. and E. Bruera (2014). “Role of corticosteroids for fatigue in advanced incurable cancer: is it a ‘wonder drug’ or ‘deal with the devil’.” Curr Opin Support Palliat Care 8(4): 346-351.
Yennurajalingam, S., S. Frisbee-Hume, M. Delgado-Guay, J. Bull and B. E (2012). “Dexamethasone (DM) for cancer-related fatigue: A double-blinded, randomized, placebo-controlled trial.” J Clin Oncol Suppl.
Yennurajalingam, S., S. Frisbee-Hume, J. L. Palmer, M. O. Delgado-Guay, J. Bull, A. T. Phan, N. M. Tannir, J. K. Litton, A. Reddy, D. Hui, S. Dalal, L. Massie, S. K. Reddy and E. Bruera (2013). “Reduction of cancer-related fatigue with dexamethasone: a double-blind, randomized, placebo-controlled trial in patients with advanced cancer.” J Clin Oncol 31(25): 3076-3082.
Yennurajalingam, S., J. S. Willey, J. L. Palmer, J. Allo, E. Del Fabbro, E. N. Cohen, S. Tin, J. M. Reuben and E. Bruera (2012). “The role of thalidomide and placebo for the treatment of cancer-related anorexia-cachexia symptoms: results of a double-blind placebo-controlled randomized study.” J Palliat Med 15(10): 1059-1064.
Yost, K. J., D. T. Eton, S. F. Garcia and D. Cella (2011). “Minimally important differences were estimated for six Patient-Reported Outcomes Measurement Information System-Cancer scales in advanced-stage cancer patients.” J Clin Epidemiol 64(5): 507-516.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of treating cancer-related fatigue in a patient in need thereof comprising orally administering to the patient a therapeutically effective amount of anamorelin, wherein the patient has:
a) a fatigue score on the FACIT-F subscale of ≤34;
b) a C-reactive protein concentration of ≥3 mg/L; and
c) cancer.

2. A method of increasing subjective perception of physical activity in a cancer patient in need thereof comprising orally administering to the patient a therapeutically effective amount of anamorelin.

3. The method of claim 2, comprising an increase of physical activity on the Godin-Shephard Leisure-Time Physical Activity Questionnaire.

4. A method of treating cancer-related fatigue in a patient in need thereof comprising orally administering to the patient a therapeutically effective amount of anamorelin.

5. The method of claim 4, comprising treatment of cancer-related fatigue on the FACIT-F subscale.

6. The method of claim 1, wherein the patient has:
a) a fatigue score on the FACIT-F subscale of ≤34; or
b) a C-reactive protein concentration of ≥3 mg/L.

7. (canceled)

8. The method of claim 1, wherein the patient has:
a) a fatigue score on the FACIT-F subscale of ≤34;
b) a C-reactive protein concentration of ≥3 mg/L;
c) cancer;
d) no evidence of depression; and
e) a hemoglobin level ≥9 g/dL.

9. The method of claim 1, wherein the patient has:
a) a fatigue score on the FACIT-F subscale of ≤30, 26, 22, or 18; or
b) a C-reactive protein concentration of ≥5 mg/L; 10 mg/L, 15 mg/L, or 20 mg/L.

10. The method of claim 1, wherein the patient has:
a) a fatigue score on the FACIT-F subscale of ≤30, 26, 22, or 18; and
b) a C-reactive protein concentration of ≥5 mg/L; 10 mg/L, 15 mg/L, or 20 mg/L.

11. The method of claim 1, wherein the patient has a C-reactive protein concentration of ≥10 mg/L.

12. (canceled)

13. (canceled)

14. (canceled)

15. (canceled)

16. The method of claim 1, wherein the patient has a hemoglobin level of greater than 9, 10, 11, or 12 g/dL.

17. The method of claim 1, wherein the method achieves a clinically meaningful improvement on the FACIT-F subscale.

18. The method of claim 1, wherein the method achieves a clinically meaningful improvement on the Godin-Shephard Leisure-Time Physical Activity Questionnaire.

19. The method of claim 1, wherein the method achieves a clinically meaningful improvement on a scale selected from PROMIS-fatigue, MF SI-SF-general, and FAACT-anorexia.

20. (canceled)

21. (canceled)

22. The method of claim 1, wherein the patient has an unintentional weight loss of from 2% to 15% within the previous 12 months.

23. The method of claim 1, wherein the patient has an unintentional body weight loss of less than 5%, 4%, or 3% in the previous 12 months.

24. The method of claim 1, wherein the patient has a BMI greater than 20 kg/m²,

22. 5 kg/m², or 25 kg/m².

25. (canceled)

26. The method of claim 1, wherein the patient has:
a) an albumin concentration in the normal range of from 3.4 to 5.4 g/dL,
b) a fasting plasma glucose concentration of from 70 to 140 mg/dL; and/or
c) an IGF-1 concentration of from 75 to 150 ng/mL.

27. The method of claim 1, wherein the patient lacks a cause of increased CRP selected from an infection or an autoimmune disorder.
